(19) United States
(12) Patent Application Publication
(10) Pub. No.: US 2008/0275432 A1
(43) Pub. Date: Nov. 6, 2008

(54) Photodynamic foam composition and sclerosis treatment

(75) Inventors: Danilo Castro, Montevideo (UY); Wolfgang Neuberger, Labuan (MY)

Correspondence Address: BOLESH J. SKUTNIK;CERAMOPTEC INDUSTRIES, INC., 515 SHAKER RD., EAST LONGMEADOW, MA, 01028, US

(73) Assignee: CeramOptec Industries, Inc.

(21) Appl. No.: 11/800,400

(22) Filed: May 4, 2007

(51) Int. Cl. A61B 6/00; A61K 31/407; A61K 47/26

(52) U.S. Cl. 606/3; 514/410; 514/777

(57) ABSTRACT

A photodynamic composition and a method for in vivo photonic treatments that is minimally invasive, versatile and precise are described. The invention allows for photonic treatments with only minimal insertions into the area of treatment, often a single one. The invention may be used with a standard insertion component making the system inexpensive and easy for doctors to use. The invention has applications in several areas of treatment. In vivo treatment of aesthetic skin blemishes such as varicose veins can be performed with minimal external effects. A predetermined amount of a photodynamic composition, as a foam, is injected into the vein or structure of concern. The composition is a sclerosis foam including a photosensitizer. By external compression, where applicable, the photodynamic composition is forced to remain in the vein or structure. After a predetermined time, radiation of appropriate wavelength from a light source is delivered directly to the vascular structure. Among the key benefits of the present invention are the elimination of targeted varicose veins, without need for anesthesia along the length of the vein; no edema; no skin reaction; and tactile appreciation.

### BACKGROUND OF THE INVENTION

1. Field of the Invention

The present invention relates to photodynamic compositions primarily to the field of phlebology, and, in particular, relates to photodynamic laser treatment to eliminated varicose veins, reticular veins and spiders veins, etc., without the need for anesthesia along the veins to be treated.

2. Invention Disclosure Statement

Varicose veins are a common condition in the entire world, affecting up to 60 percent of all older people, in particular, especially older women, who are more likely than men are to have this problem.

The desire of people to correct vein enlargement has both a medical and a psychological basis. Varicose veins can cause aching pain and discomfort. Sometimes the condition leads to more serious health problems. Varicose veins may also signal a higher risk of other disorders of the circulatory system. Further, varicose veins on the legs are particularly upsetting and prevent many people from exposing these conditions in public.

Signs and symptoms of varicose veins may include achy or heavy feeling in the legs, and burning, throbbing, muscle cramping and swelling in the lower legs. Prolonged sitting or standing tends to make the legs feel worse. This may lead to frequent itching around one or more of the veins; possible skin ulcers near the ankles, which represent a severe form of vascular disease and require immediate attention. Varicose veins are dark purple or blue in color and may appear twisted and bulging—like knots in a cord. They commonly appear on the backs of the calves or on the inside of the legs. But, they can form anywhere on the legs, from the groin to the ankle.

Spider veins are similar to varicose veins, but they're smaller. Spider veins are found closer to the skin's surface and are often red or blue in color. They occur on the legs, but can also be found on the face and on the nose. Spider veins vary in size and often look like a spider's web or a tree branch.

Other types of varicose veins include: Venous lakes that are pools of blood in the veins, often found on the face and neck. Reticular veins are flat and blue veins under the skin and often appear behind the knee. Telangiectasias are fine clusters of blood vessels similar to spider veins, reddish in color and often found on the face or upper body. Occasionally, veins deep within the legs become enlarged. In such cases, the affected leg may swell considerably and may or may not be accompanied by pain and redness. This warrants urgent medical attention as it may indicate a blood clot—a condition known medically as thrombophlebitis.

Historically, different methods have been developed to treat this problem and billions of dollars are being spent annually by people to remove varicose veins. The first treatment to remove varicose veins was sclerotherapy and beginning in the Twentieth century surgery was the best solution to eliminate varicose veins. A relatively new development is Endovascular Laser Surgery (ELVeS™) and is an effective solution instead of surgery. In sclerotherapy, the procedure involves the injection of a sclerosing agent into the small or medium-sized varicose veins which scars the veins. The process closes the veins forcing the blood to reroute to healthier veins. In six or seven weeks, the treated varicose veins should fade. Although the same vein may need to be injected more than once, sclerotherapy is effective if done correctly, normally only in small veins. In addition, a new and improved type of sclerotherapy, called micro-sclerotherapy, uses improved solutions and injection techniques that increase the success rate for removal of spider veins. Sclerotherapy doesn't require general anesthesia and can be done in the doctor's office, with only local anesthesia of the treatment area.

Some of the procedures used to treat varicose veins are as follows:

(1) Catheter-assisted procedures. In this treatment, the doctor inserts a thin tube (catheter) into an enlarged vein and heats the tip of the catheter. As the catheter is pulled out, the heat destroys the vein by causing it to collapse and seal shut. This procedure is usually done for larger varicose veins. Other catheter-assisted methods use a blade to destroy varicose veins or radio waves to close them.

(2) Vein stripping. This procedure involves removing a long vein through two small incisions. The vein is tied off and removed by pulling it out. This procedure requires general anesthesia and hospitalization with a lengthy recover period and leaves scarring.

(3) Ambulatory phlebectomy. The doctor removes smaller varicose veins through a series of tiny skin punctures. A local anesthesia is used in this outpatient procedure. Scarring is generally minimal.

(4) Endoscopic vein surgery. This procedure is required in advanced cases involving leg ulcers. The surgeon uses a thin video camera inserted in the leg to visualize and close veins. Only small incisions are needed.

(5) Laser surgery is used to close off smaller varicose veins and spider veins, especially on the upper body and the face. In the past, varicose veins in the legs didn't respond consistently to laser treatments, especially the larger veins, and some doctors doubted whether laser surgery actually worked. Now, however, new technology in laser treatments can effectively treat varicose veins in the legs using the endovenous laser vein system ELVeS. In this procedure, a local anesthesia is applied and an optical fiber is inserted into the vein. Effective laser radiation, typically 980 nm, 930 nm or 810 nm radiation, applies thermal energy to the vein as the optical fiber is withdrawn resulting in vein closing and the vein eventually disappearing through absorption.

Laser treatment below the surface of the skin has also been described in U.S. Pat. No. 5,578,029 to Trelles et al. There a method is described whereby an optical probe is inserted into the skin adjacent to a vascular abnormality. Introducing laser pulses will serve to collapse and close off the vein. This method requires multiple insertions of the device along the desired vein to treat the abnormality since the probe is inserted perpendicular to the axis of the vein. The procedure has limitations. First, the described method is specific for treatment of veins, primarily in the leg, and is therefore limited to that specific treatment. Second, the procedure requires a low power beam to illuminate and direct the probe to the treatment site under the skin. Treatment is then limited to underskin sites at a depth that the illumination beam can penetrate. Third, the method describes delivery of the laser power to the vicinity of, and outside the vein requiring treatment. Therefore, it may be difficult for users to have on hand all the various sized probes needed for different applications; and it may be costly for them to obtain additional attachments. Second, the special probes used in the treatment are too expensive to be disposed of; therefore they must be sterilized and sharpened after applications. This is both time consuming and cumbersome for the user. Time and effort are required to ensure that each probe is properly sterilized; and suitable sharpening depends on the care and skill of the user. Such laser treatment closes off the vein by collapsing its wall, an indirect solution.

The method also requires multiple insertions of the device into the patient's skin. Each insertion must be made so that the probe is placed in close proximity to the vein being treated. The treated section of the vein is closed off, the device is removed, reinserted into another section of the vein, and the lazing procedure is repeated. This requirement makes the device more difficult to use. Further, by introducing multiple punctures of the skin the risk of infection is increased, as is the chance for disfiguration of the skin surface.

Another example of treating varicose veins is illustrated by U.S. Pat. No. 6,200,332 by Del Giglio. The device of Del Giglio allows a simple, single insertion per treated structure and specific laser delivery. The needle is inserted into the vascular structure and any abnormalities are eradicated starting from the source and continuing through the entire structure. Third, when coupled with x-ray imaging, the present invention may be used to treat various internal body structures for example during surgery. X-ray imaging allows the user to orient the device within the body structures. Laser delivery treatment can then be administered as described above.

An aim of the present invention is to provide a method and device to safely and effectively treat underskin abnormalities without incurring the problems and deleterious side effects associated with the prior art. Additionally the present invention aims to provide a new composition of photosensitizer and components especially suitable for medical applications such as treatment of underskin abnormalities.

### OBJECTS AND SUMMARY OF THE INVENTION

It is an objective of the present invention to address the need for an effective photonic treatment that can be used to eradicate vascular abnormalities with greater specificity and efficiency, and minimal invasiveness.

It is another objective of the present invention to provide a method of varicose vein sclerosis treatment which is more effective on essentially tortuous varicose veins secondary to Saphenous veins and reticular veins to about 2 mm diameter or less.

It is yet another objective of the present invention to provide a method which will work with a photodynamic sclerosis composition in the vein.

It is a further objective of the present invention to provide a photosensitizer foam formulation for PhotoDynamic Therapy treatments, especially for treatment of vascular and other underskin abnormalities Briefly stated, the present invention describes a photodynamic composition and a method for in vivo photonic treatments that is minimally invasive, versatile and precise. The invention allows for photonic treatments with only minimal insertions into the area of treatment, often a single one. The invention may be used with a standard insertion component making the system inexpensive and easy for doctors to use. The invention has applications in several areas of treatment. In vivo treatment of aesthetic skin blemishes such as varicose veins can be performed with minimal external effects. A predetermined amount of a photodynamic composition, as a foam, is injected into the vein or structure of concern. The composition is a sclerosis foam including a photosensitizer. By external compression, where applicable, the photodynamic composition is forced to remain in the vein or structure. After a predetermined time, radiation of appropriate wavelength from a light source is delivered directly to the vascular structure. Among the key benefits of the present invention are the elimination of targeted varicose veins, without need for anesthesia along the length of the vein; no edema; no skin reaction; and tactile appreciation.

The above and other objects, features and advantages of the present invention will become apparent from the following description read in conjunction with the accompanying drawings, in which like reference numbers in different drawings denote like items.

### BRIEF DESCRIPTION OF THE FIGURES

FIG. 1 illustrates the process of temoporfin interacting with cells of the vein wall.

FIGS. 2A to 2C illustrate how to perform sclerosis treatment: with a needle to introduce the sclerosing product into varicose veins (A), later produce a fibrotic cord (B), and in 45 days the fibrotic cord disappears (C).

FIG. 3A and 3B illustrate different foam and bubble formations.

FIGS. 4-E/(1A to 1L) illustrate a sequence of photographs of the treatment of a varicose vein in a lower leg; Photograph 1A and 1B illustrate the exterior view of the vein; Photograph 1C illustrates the application of the photodynamic sclerosis composition and the leg after; Photograph 1E and 1F illustrate the application of the laser radiation to the leg; Photographs 1G,. 1H, 1I and 1J illustrate the rise in temperature of the treatment area after the application of the laser radiation; and Photographs 1K and IL illustrate the same area 7 days after treatment showing the disappearance of the vein and the lack of other complicating medication conditions on the leg.

FIGS. 5 A-E/(2A to 2D) illustrate a sequence of photographs of the treatment of a varicose vein behind the knee; Photograph 2A is a before picture of the rear knee area; Photograph 2B is a picture showing the insertion of the photodynamic sclerosis composition; Photograph 2C shows the application of laser radiation; and Photograph 2D illustrates the same area after treatment.

FIGS. 6A to 6B illustrate the removal of a treated vein and the vein by itself for use in a histological study.

### DETAILED DESCRIPTION OF PREFERRED EMBODIMENTS

Many women in particular, suffer, have suffered or are going to suffer at some time with varicose veins in their life. It is an esthetic pathology and it has important pathological general connotations.

It is a problem that belongs to mostly women, in general, and normally women between 25 to 40 years old especially after child birth; this results from a hormonal point of view from an increase of estrogen and progesterone that reduce the vascular compliance, especially in veins that don't have a muscle wall well defined. The veins in this situation are more prone to ecstasies with dilated walls and result in varicose veins. Further, the expanding uterus compresses the pelvic veins and reduces good blood return placing additional stress on the lower leg veins. This dilation also results in varicose veins.

Varicose veins cause legs aesthetical alterations and this modifies the human behavior, especially in women. There are women thus who don't go to the beach or don't use short skirts due varicose veins in legs.

There is not an adequate definitive solution, just medical, functional and cosmetic treatments that in general do not achieve the patient's complete satisfaction.

The goals of the present invention in regards to the treatment of varicose veins are to: (1) provide a medical method to eliminate bulk varicose veins; (2) provide a process for destruction of the endothelium cells and the muscular layer that were previously altered with sclerosing foam; and (3) increase the Phagocytes and Polymorphonuclear leukocytes arrivals to eliminate finally the varicose vein.

When the photosensitizer Temoporfin is activated with light from a diode laser, e.g., 625 nm, this produces an intracellular oxidation that acts to modify the cell membrane's properties, the cytoplasm, ribosomes, Golgi's apparatus, and nucleus, eventually triggering a series of events that result with the cell apoptosis.

The cells when exposed to the effects of Temoporfin begin a series of morphologic changes. The plasma membrane alters and the characteristic blebbing appears. The cell volume decreases considerably and the cytoplasm condenses. The nucleus becomes smaller and chromatin become denser and eventually collapses splitting into several spheres of material.

At the end of apoptosis, the cell is ingested by phagocytosis by phagocytes or by nearby cells avoiding the inflammatory response in necrosis. Even though the cell disappears, there is an increase of the collagen web. This improves the support of collagen, realignment of the collagen fibers and elastin, decreases the gelatinous consistency of the fundamental inter-cell substance, improves oxygenation and cell nutrition, and decreases toxic metabolites' retention and edema.

Temoporfin is a very efficient photochemical generator of activated triplet oxygen which does not require a large dose of the drug to kill cells nor a long exposure to light.

In the photodynamic process, activated Temoporfin, in the presence of oxygen produces: 1) an instantaneous hyper oxygenation and increases venous endothelium destruction. This increases vein collapse and primary obstruction; 2) reduces blood catching and micro clot retention due to quickly formed primary obstruction; 3) creates fibrous tissue to produce a more firm skin structure from 7 to 10 days and therefore the final sclerosis is finished in no more than 21 days after treatment by the present invention; 4) PMN arrives before the body detects the varicose veins as a foreign object and the final fibrous elimination is precocious and complete.

Exterior characteristics make this treatment ideal for medical eradication of varicose veins. These characteristically make this therapeutic option ideal for use in bulk varicose veins sectors when patients refuse surgery.

This method is relatively straight forward in actual practice. A butterfly is used for canalizing the varicose veins and introducing the photodynamic composition slowly. After a short period of time of about 15 minutes which allows the photodynamic composition to enter into the vein walls, the laser radiation is applied to the area having an energy density of about 20-28 J/cm2.

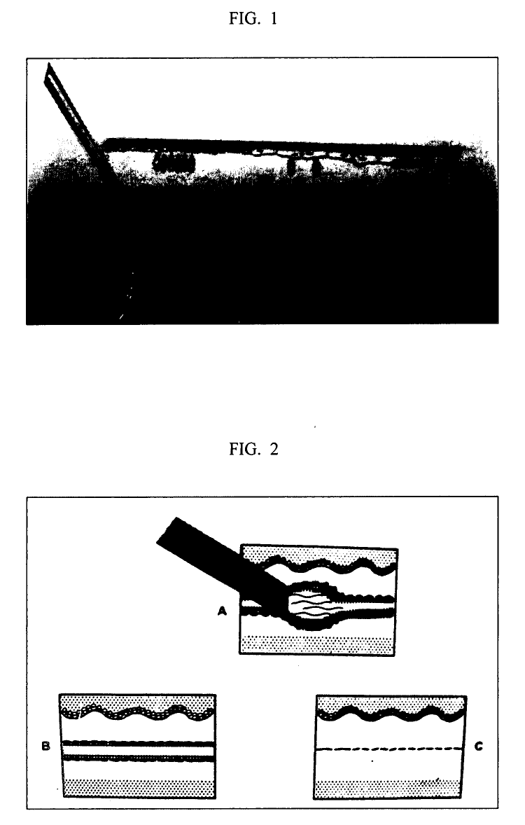

FIG. 1 illustrates the process of temoporfin interaction upon activation leading to the intracellular oxidation responsible for the alterations of the wall cell membrane's surface and the nuclear, the mitocondrias, Golgi's appliance, the endoplasmic reticulum and the ribosomes resulting in the death of the cells or cell apoptosis.

Temoporfin is totally inactive in the dark and is activated with low intensities of light turning it into a powerful isolated-oxygen generator.

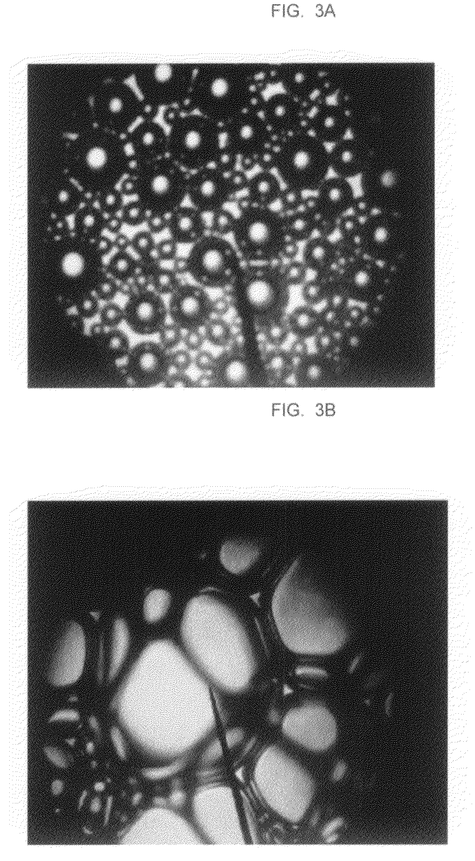

As seen in FIGS. 2A to 2C, sclerotherapy starts with the injection of drugs, FIG. 2A, capable of transforming the wall of a varicose vein into a fibrotic cord. FIG. 2B. After approximately 45 days the fibrotic cord disappears. FIG. 2C

The sclerosing foam (SF) is a mixture of gas and a liquid solution with tensioactive properties; the bubble size should be preferably under 100μ. The foam forms a coherent bolus inside the vein that prevents any mixing of the drug with the blood.

The photodynamic sclerosis composition/foam (PDSF) increases the wall transformation into a fibrotic cord due to the increase varicose intima destruction and posterior cicatrization cord.

With the photodynamic sclerosing foam (PDSF), there is full control of the drug concentration inside the vein, improved time of contact between the sclerosing agent and the endothelium, increased intima destruction and more quickly transforms the varicose veins in a fibrotic cord and its posterior elimination by phagocytosis.

Accelerating permanently the elimination with an injection into the saphenous trunk together with the tributaries at low costs, no hospitalization, and no anesthesia, has improved the sclerosis foam's (SF) use.

The development of medical foam was initiated in 1993 by Juan Cabrera, a 05/03/07 vascular surgeon from Spain, who proposed the use of a therapeutic foam consisting of polydodecanol [alternatively, dodecyl-polyethylene-glycol-ether; hydroxyl polyethoxy dodecane](POL) in the treatment of varicose veins. This was a true step forward in the treatment of superficial venous insufficiency. In 1997 Alain Monfreux reported a technique utilizing a glass syringe and a sterile plug to produce a weak foam. Patrick Benigni and Symon Sadoun produced a POL foam with a disposable syringe and a tap. In 1999 Mingo-Garcia reported another technique using helium and a specially designed device for the application of the foam. In the 2000 Lorenzo Tessari presented his three-way tap technique which was capable of extemporarily preparing a very good foam at an extremely reduced cost. To produce Tessari's foam, a three-way stopcock is needed, coupled with a 2.5 mL syringe filled with 1 cc of a drug and a 5-mL syringe with 4 to 5 mL of atmospheric air. Twenty quick passages of the solution are made. After the first 10 passages the tap is narrowed as much as possible. This will form a high-quality and high-consistence foam.

The present invention using the photodynamic sclerosis foam blends a small amount of POL, from 0.5 to 1.5% by volume, with a 50% glucose solution and 50 to 100 ng/ml of Temoporfin (photosensitizer) present in a liposomal solution. The glucose improves the foam's consistency and greatly enhances the activated photosensitizer fibrotic cord formation. Both liposomal and straight temoporfin have been prepared in the foam composition/formulation. Prior art indirectly predicted that such foams would be difficult to form stably and thus use in photodynamic therapy could be marginal at best.

### Sclerosing Foam and Photo Dynamic Sclerosing Foam

Sclerotherapy is the injection of drugs capable of transforming the wall of a varicose vein into a fibrotic cord. The end point of sclerotherapy should be permanent occlusion, but this does not always occur with liquid sclerosants. The main factor for insufficient sclerotherapy is represented by the volume of blood in which the drug will be diluted and the rapidity that it produces its effects.

With liquid sclerosants the injection inside a vein segment raises the inner drug concentration to a peak, followed by a blood dilution and quite rapid decrease in the inner concentration of sclerosant. The shape of the curve is ruled by the speed of injection, the ratio injected volume to size of the vessel, and by the blood flow. Sclerosis will be triggered only if a threshold level of drug concentration occurs or if there is a minimal effective concentration for a sufficient period of time.

In telangectasia we can expect a straight rise and a relatively long plateau, where only the drug will be present inside the telangectasia.

In a large great saphenous vein (GSV) with significant reflux, the peak will be reached slower than in the previous example and will be related to the size of the needle and to the fluidity of the injected material. The maximum concentration of the sclerosant in that vein segment will be related to the volume of blood with which it will be diluted. This could explain why sclerotherapy has never been problematic in terms of drug power for telangectasia, and why saphenous sclerosis has always been difficult to achieve.

When foam is injected it forms a coherent bolus inside the vein. Due to its properties, this bolus has controlled and uniform properties and can thus be controlled in situ for a definite time. This will lead to optimal, and for the first time controlled, sclerosis.

Foam is a nonequilibrium dispersion of gas bubbles in a relatively small volume of liquid which contains surface active macromolecules (surfactants). These preferentially adsorb at the gas/liquid interfaces and are responsible both for the tendency of a liquid to convert into a foam and for the stability of the produced dispersion. The sclerosing foam is a mixture of gas and a liquid solution with tensioactive properties, and the bubble size should be preferably under 100 μm. The behavior of the sclerosing foam is different when injected, compared with the action of a liquid solution. The active substances, POL and Temoporfin, have more time in contact with the intima vein surface. Not only is the Photodynamic action more intense but also the hydrophobic photosensitizer, Temoporfin, penetrates deeper after the sclerosing action, due to these the last detergent makes endothelial damage through interference with the cells' surface lipid. Detergent produces maceration of the endothelium within 1 second exposure, intercellular cement is disrupted, causing desquamation of the endothelial cells.

The photosensitizer, Temoporfin, benefits from this action to penetrate deeper in the endothelium and places it just near the muscular layer of the vein. When Temoporfin is activated by photonic radiation, it produces in the presence of oxygen a higher hyper oxygenation which will result in cell destruction and immediately begins the reconstruction through the fibroblast cells that increase in it as much just inside differentiation and multiplication as fibroblast for endo chemistry factors.

The most common mistake with foam is to consider it as a single entity. In fact, according to the method chosen, it is possible to produce very different foams, with different characteristics, complication rates, and therapeutic indications. We can classify foams by bubble diameter (froth, foam, minifoam, and microfoam), or by the relative quantity of liquid (the shape is the result of the competition between surface tension and interfacial forces) as wet foam (nearly spherical bubbles—wetness, or the volume fraction of liquid is over 5%). The wet foam is shown in FIG. 3A. The dry foam (polyhedral bubbles—the volume fraction of liquid is below 5%) is shown in FIG. 3B.

Wet foam has maximum stability, because when the bubble is polyhedral, as in dry foams, there is greater competition between surface tension and interfacial forces. Uniform diameters also mean more stability, because smaller bubbles empty into larger ones according to Laplace's law, because for smaller diameters there will be a higher internal pressure. Extemporary sclerosing foams, like Monfreux's foam, often have a two-stage behavior, acting as dry foam with polyhedral bubbles in the very first moments after generation then, when dissolution of bubbles creates a wetter environment, the foam has spherical bubbles. More standardized sclerosing foam (e.g., Tessari's foam) appears to be wet even in the initial stages. This produces more stability and uniformity. Another way to classify foam is considering the standard of production: it can be low-or medium-grade for extemporary foam, but is maximal only for industrial high-standard foam. Even when it seems very stable, foam is always in evolution between the different foams.

A sclerosing foam shows peculiar properties: adhesiveness and compactness (with the possibility of manipulating the foam after injection and displacing effect on blood), syringe ability(or ability to be injected with a small needle without losing its characteristics), greater volume for the same quantity of liquid agent (possibility of treating longer vein segment), long duration (long enough for therapeutic action), enhanced spasm generation (less risk of blood collection inside the sclerosed vein), echo visibility, enhancement of sclerosing power with reduced drug dose and concentration, and selectivity of action on endothelium (lesser risk in case of extravasation). These foam actions were found to be good in combination with the sensitizer (Temoporfin), the foam is selective and adhesive for a long duration for endothelium action, and this action helps Temoporfin penetrate into the vein wall and makes it a better peeper action. Temoporfin's action helps the sclerosing foam too. After activation the cells' destruction allows the sclerosing foam to complete in a short time the spasm generation and close definitively the vein. Stability of the temoporfin solution in the sclerosing foam was predicted to be limited and thus the good activity observed, was unexpected to those skilled in the art.

### EXAMPLE 1

This patient, a 64 year old male, was surgically intervened twice in each leg in the past and consulted for varicose veins secondary to insufficient perforates veins.

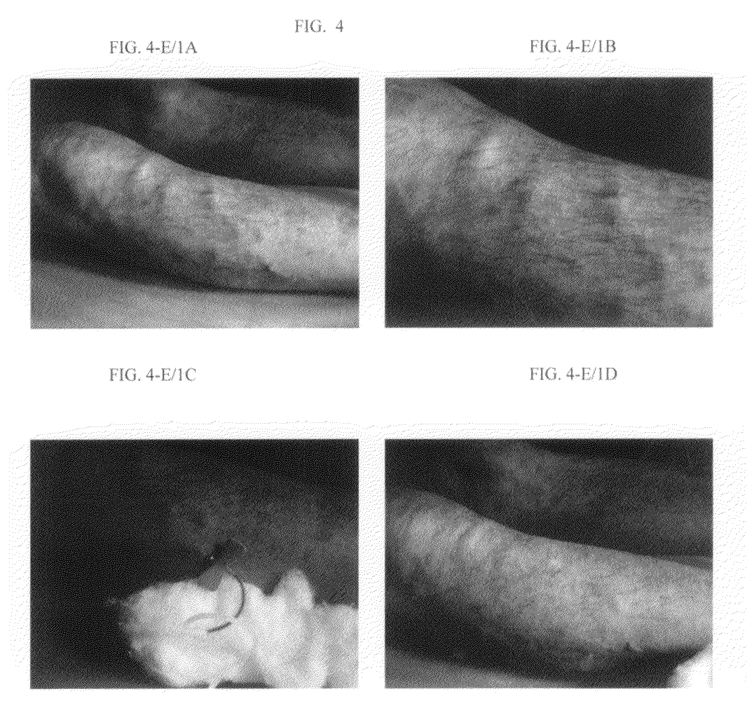

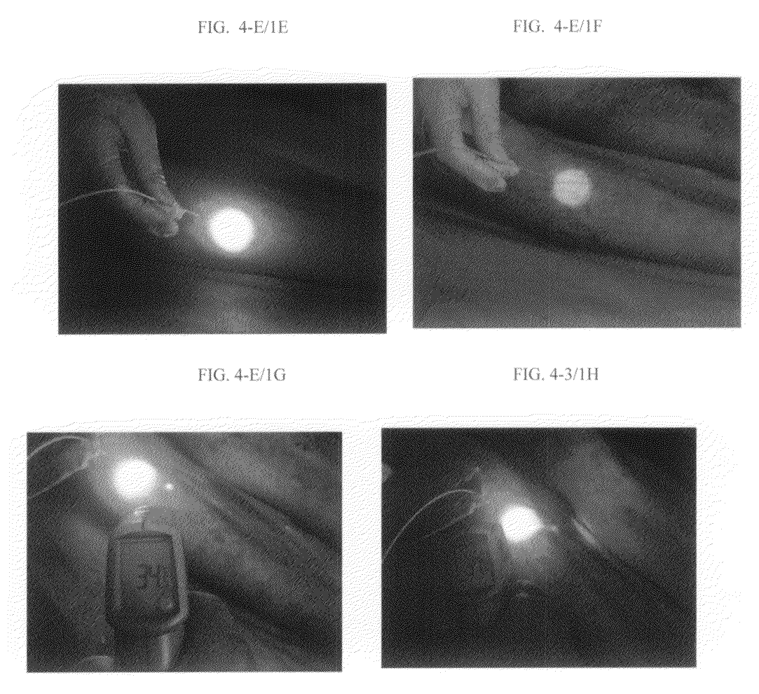

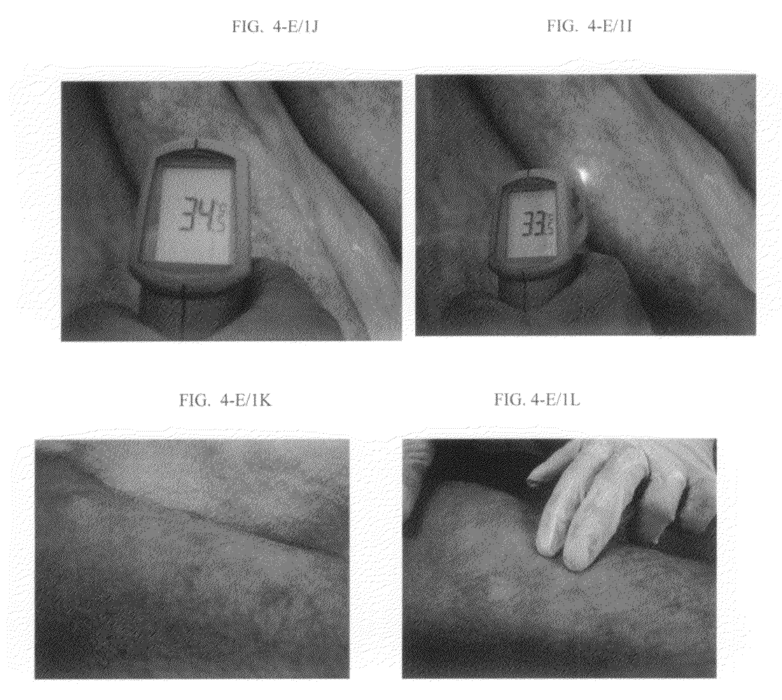

FIGS. 4-E/(1A to 1B) illustrate that before treatment it is possible to observer varicose vein formation in the external surface of the leg below knee. They provide important trajectories visible with minimal effort. The particular sclerosing foam used in this Example was prepared with polydodecanol 1% (detergent solution) plus 30% Glucose solution and 100 ng/ml of Temoporfin.

FIG. 4-E/(1C to ID) illustrate the application of the photodynamic sclerosis foam. A butterfly is used for introducing the photodynamic sclerosing foam. Additionally, when necessary, ultrasound guidance was used for implant infusion catheter positioning.

FIG. 4-E/(1E and IF) shows the application of laser radiation, 652 nm, for the activation of Temoporfin: the drug light interval was 15 minutes and the final light energy is about 16 Joules/cm2. This dose has been well tolerated without side effects and is sufficient for a good treatment with this PDF.

In order to judge the activation of temoporfin is to track the temperature increase in the area of treatment as shown in the pictures. In these pictures, FIG. 4-E/(1G and 1H), we can see the temperature at the start of activation of 34.5° C. and the temperature after activation of 37° C. This temperature increase with PDS activation of 2.5° C. is the external expression of the temoporfin activation.

In the next pictures, FIG. 4-E/(1I to 1J) after 15 minutes after laser activation there is a continued 1° C. temperature increase.

FIGS. 4-E/(1K to IL) show the evolution of the area of treatment post PDS 7 days: the varicose vein has disappeared and there is no fibrous cord. There is no ecchymosis, hematomas, and hyper pigmentation.

### EXAMPLE 2

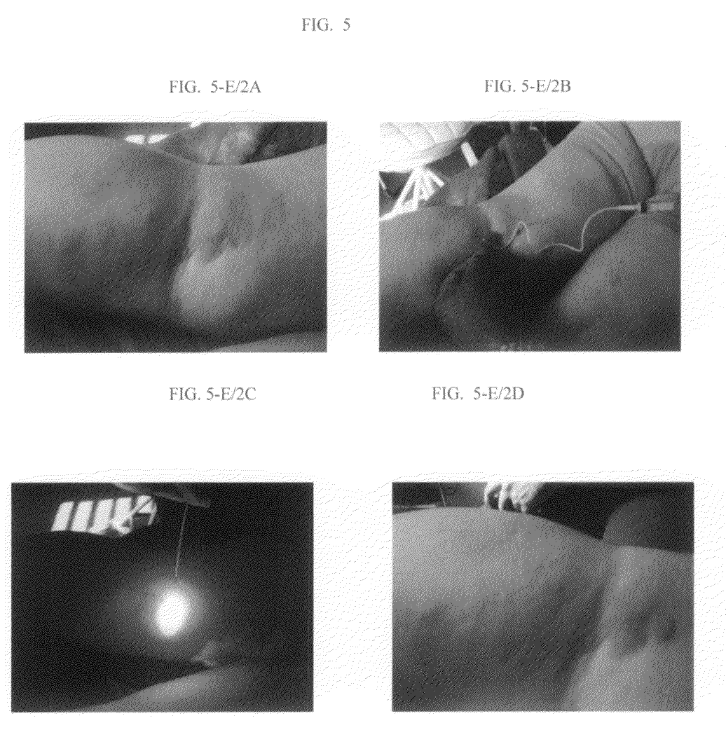

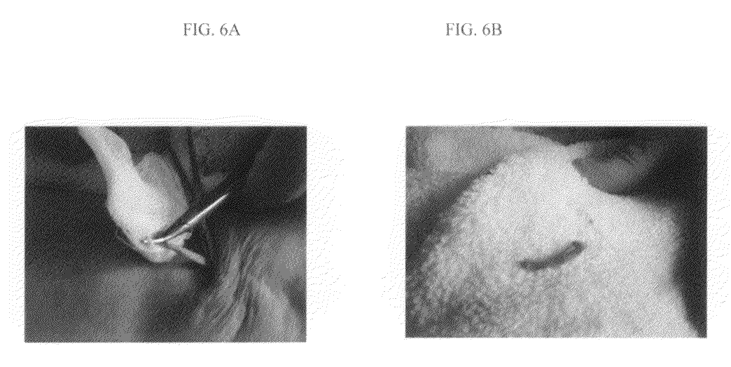

In FIG. 5-E/(1A and 1B), a 70 year old woman, has laser endovascular treatment 15 days before, but now PDS Photo Dynamic Sclerosis is applied behind the knee: The photodynamic sclerosis foam used was Polydodecanol 1.25% plus Foslip 50 ng/ml.

FIG. 5-E/(2C and 2D) shown laser activation: at an energy density of 20 J/cm2. The vein was removed for histological study.

Although the above Examples use the photosensitizer with a foam for forming a bolus in order to prevent the flow of the photodynamic sclerosis composition through the veins of concern, it is possible to not use the foam but apply a blocking means to the one or more veins to prevent the flow of the sensitizer by itself through the vein until after the treatment is provided. The blocking means may be external compression, a balloon applied by catheter or a previously collapsed vein section.

Not only in legs are there varicose veins, but they are in the esophagus mucosa where treatment is difficult and there is a danger of bleeding during treatment. The past treatment has been by sclerosis therapy, but PDS sclerosis should be more effective and quicker, two essentially conditions to obtain good results.

Having described preferred embodiments of the invention with reference to the accompanying drawings, it is to be understood that the invention is not limited to the precise embodiments, and that various changes and modifications may be effected therein by one skilled in the art without departing from the scope or spirit of the invention as defined in the appended claims.

What is claimed is:

1. A method for in vivo photonic treatments of vascular structures using a light source and a photodynamic sclerosis composition, comprising the steps of:
a. identifying one or more veins in an area of treatment;
b. introducing a photodynamic sclerosis composition into said one or more veins;
c. awaiting a sufficient time to allow said photodynamic sclerosis composition to interact with said one or more veins; and
d. applying radiation from an appropriate light source, emitting an activation wavelength absorbed by said composition, to said area of treatment for a sufficient time to activate a photosensitizer in said composition.

2. The method for in vivo photonic treatments according to claim 1, further comprising the step of:
e. monitoring a temperature of said area of treatment to insure an adequate activation of said photosensitizer.

3. The method for in vivo photonic treatments according to claim 1, wherein said one or more veins are selected for the group varicose veins, spider veins, reticular veins.

4. The method for in vivo photonic treatments according to claim 3, wherein said one or more veins are varicose veins.

5. The method of in vivo photonic treatment according to claim 1, wherein said photodynamic sclerosis composition is a foam.

6. The method of in vivo photonic treatment according to claim 1, wherein said radiation is from a laser source operating at a wavelength of about 652 nm.

7. The method of in vivo photonic treatment according to claim 5, wherein said foam has bubble diameters of less than or about 100 microns.

8. A photodynamic composition for use in an in vivo method such as claim 1, comprising:
a. a foam; and
b. a photosensitizer or a photosensitizer precursor.

9. The photodynamic composition, for use in the in vivo method of claim 1, according to claim 8, wherein said foam is a sclerosis foam.

10. The photodynamic composition according to claim 9, wherein said sclerosis foam comprises a detergent and a glucose solution (glucose solution).

11. The photodynamic composition according to claim 10, wherein said detergent is polydodecanol.

12. The photodynamic composition according to claim 8, wherein said photosensitizer or photosensitizer precursor is temoporfin.
